(19) United States
(12) Patent Application Publication
(10) Pub. No.: US 2025/0221620 A1
(43) Pub. Date: Jul. 10, 2025

(54) APPARATUS AND METHOD FOR ATTACHING A HANDS-FREE LENS TO A MICROSCOPE FOR USE DURING OCULAR SURGERY

(71) Applicant: OCULUS SURGICAL, INC., Port St. Lucie, FL (US)

(72) Inventors: Michael ANNEN, Ft. Pierce, FL (US); Steffen ADAMOWICZ, Solms (DE); William O'BRIEN, Port St. Lucie, FL (US)

(21) Appl. No.: 18/848,353

(22) PCT Filed: Mar. 17, 2023
(86) PCT No.: PCT/US2023/015489

§ 371 Date: Sep. 18, 2024

Related U.S. Application Data
(60) Provisional application No. 63/321,285, filed on Mar. 18, 2022.

(51) Int. Cl. A61B 3/13

(52) U.S. Cl. CPC A61B 3/13

(57) ABSTRACT

An apparatus is presented for attaching a lens to a micro-scope with an optical attachment. The apparatus includes the lens with one or more translational degrees of freedom such that the lens is configured to translate along one or more directions relative to the microscope and/or one or more rotational degrees of freedom such that the lens is configured to rotate about one or more rotational axes relative to the microscope. A system is also presented that includes the apparatus and the optical attachment. The optical attachment includes a lens arm and/or the viewing attachment configured to move the lens and the lens arm relative to the microscope. A method is also presented for using an optical attachment to position a hands-free lens relative to a microscope.

### CROSS-REFERENCE TO RELATED APPLICATIONS

This application claims priority to U.S. Provisional Application No. 63/321,285, filed Mar. 18, 2022, under 35 U.S.C. § 120.

### BACKGROUND

While observing the eye of a patient under a microscope, medical professionals may place an additional optic in contact with the cornea to improve their view of an intraocular structure. Various optics are known which accommodate an ophthalmologist's view of an eye in different ways. For example, a gonioscopy lens provides an ophthalmologist with an angled view through the cornea that allows visualization of the peripheral sections of the anterior chamber that are otherwise difficult to visualize.

### SUMMARY

Primary Open Angle Glaucoma is a disease state characterized by elevated intraocular pressures, the cause of which is most commonly attributed to a restricted outflow pathway through the trabecular meshwork and Schlemm's canal. These anatomical structures are located within the iridocorneal angle 901 (see FIG. 9A) in the periphery of the anterior chamber. When surgical intervention is required to increase aqueous outflow, precise visualization of these fine structures is needed. The iridocorneal angle is normally not visible from the eye's exterior due to an optical phenomenon known as Total Internal Reflection (TIR). FIG. 9B is a ray diagram that illustrates an example of light refraction and total internal reflection at a boundary between materials 903, 905 of different index of refraction n₁, n₂ where n₁ is greater than n₂. In one embodiment, the first material 903 is the eye (e.g. shown in the left side of FIG. 9B, an incident ray from within the eye is incident at the eye-air interface at a first angle φ₁ (e.g. measured relative to a normal 907 to the interface) and is refracted as a refracted ray at a second angle φ₂ (e.g. also measured relative to the normal 907) that is greater than the first angle φ₁ . As shown in the middle of FIG. 9B, an incident ray from within the eye is incident at the eye-air interface at a first angle φ₁ (e.g. greater than the first angle φ₁ in the left side of FIG. 9B) and is refracted along the boundary of the eye-air interface (e.g. the second angle φ₂ is 90 degrees). The first angle φ₁ in the center of FIG. 9B is known as the “critical angle” since incident light from within the eye on the eye-air interface is refracted along the eye-air interface. As shown in the right side of FIG. 9B, an incident ray from within the eye is incident on the eye-air interface at a first angle φ₁ that is greater than the critical angle and the incident ray is reflected (TIR) at the eye-air interface back into the eye at a second angle φ₂ that is equal to the first angle φ₁. As shown in the right side of FIG. 9B, TIR occurs at a boundary between the two materials 903, 905 with a difference in index of refraction, when the incident angle φ₁ exceeds the critical angle (center of FIG. 9B). If the ray approaches such a boundary at a shallow enough angle φ₁, (e.g. equal to or greater than the critical angle), it is possible that the ray exiting into the second material 905 (e.g. air) with the lower index of refraction n₂ would be refracted such that the angle of refraction theoretically would be greater than 90 degrees (e.g. when the incident angle φ₁ is the critical angle) or beyond parallel to the boundary thus becoming a reflection rather than refraction (e.g. when the incident angle φ₁ exceeds the critical angle). The critical angle for the tear-air interface is about 46°. If light from the interior of the eye strikes the cornea at an angle shallower than 46° (e.g. if the incident angle φ₁ is greater than the critical angle for the eye-air interface), TIR will occur and light will not exit the eye (FIG. 9A).

As shown in FIG. 10, a hand-held gonioscopy lens 1000 in principle acts as a continuation of the cornea and permits light from the iridocorneal angle to cross the air boundary at an angle closer to perpendicular. The gonioscopy lens 1000 includes a first surface 1003 (e.g. in contact with the eye 115) and a second surface 1005 (e.g. in contact with the air). Since the lens 1000 acts as a continuation of the cornea, and thus there is minimal difference in the index of refraction between the lens 1000 and the cornea, no TIR occurs at the lens-cornea boundary. Additionally, since the normal to the second surface 1005 is substantially aligned with the incident light within the lens 1000 to the lens-air boundary, the incident angle of the incident light on the lens-air boundary is relatively small in magnitude and thus no TIR occurs at the lens-air boundary. To avoid pockets of air between the first surface 1003 and the eye 115, generally an ophthalmic solution is applied onto the first surface 1003 prior to placing the gonioscopy lens 1000 in contact with the eye 115. The surgeon (or an assistant) holds the lens 1000 on the eye 115 This permits the surgeon to view the interior anatomical structures of the eye at the iridocorneal angle 901.

The inventors of the present invention recognized that the majority of optics used for optical procedures (e.g. a majority of gonioscopy lenses used for anterior surgical procedures) are hand-held lenses that must be manually retained in position on the cornea. In most cases, the surgeon operates with the handheld gonioscopy lens in one hand and a surgical instrument in the other. In straightforward procedures (e.g. bypass shunt placement) this is an effective way to perform the surgery as the surgeon has direct control of the view and the instrument simultaneously. In more complex procedures, limiting the surgeon to use of one hand increases the time and difficulty of the procedure. For this reason, in some cases it may be beneficial or even necessary for the surgeon to be able to operate bimanually using a second instrument. In order to do so, the handheld gonioscopy lens is generally held by an assistant with the understanding that the lens will frequently need to be repositioned through verbal instructions.

The inventors of the present invention recognized that some lenses offer self-stabilization features, e.g. a flange along the lower lens surface that extends to increase the base of the lens. The inventors recognized that while the stabilization features will improve the lens retention, adjustments of the lens will likely still be needed, requiring the surgeon to remove an instrument in order to manually reposition the lens. The inventors also recognized that a flange also presents a different issue in that it can restrict access to various insertion points. The flange may also impede visualization. The inventors of the present device realized the need for an alternative self-stabilizing lens that could be used without the aid of an assistant and enable true bi-manual surgery.

One instance of a microscope suspended gonioscopy lens has been identified in U.S. Patent Publication Number 2013/0182223. The design of this complex system centers on a counterweight style lens holder. However, the inventors of the present invention noticed drawbacks of this suspended lens design including that the lens only has one rotational degree of freedom (about one rotational axis) relative to a lens arm and attachment that is used to suspend the lens from the microscope and that the lens does not feature a translational degree of freedom relative to the attachment. Thus, the inventors of the present invention developed an improved lens arm design herein which features multiple rotational degrees of freedom (about two or more rotational axes) and multiple translational degrees of freedom of the lens and an attachment that suspends the lens to the microscope.

Another instance of a suspended gonioscopy lens has been identified in U.S. Pat. No. 8,118,431 ('431 patent hereafter). This design however specifies the attachment to the objective lens of the microscope in its description and abstract. It also focuses on using a mirrored gonioscopy lens and attachment configured to position the lens between the microscope and the eye to simultaneously view the surface and the interior of the eye (a claim taught in U.S. Pat. No. 4,157,859 to Terry, as well as in US20060098274 Kitajima). The '431 patent fails to teach how the lens is suspended from the objective lens to provide a method for compensation of patient eye movement, misalignment of the eye relative to the microscope optical axis, and the necessary safety feature to prevent patient trauma in the event of unintended large microscope movement. Thus, the inventors of the present invention developed the improved lens arm and lens design herein, to overcome these noted drawbacks in the '431 patent.

In vitreo-retinal procedures, or procedures in the posterior chamber of the eye, the inventors recognized that a wide-angle viewing attachment (“viewing attachment” herein) is often used on the ophthalmic operating microscope. A wide-angle viewing attachment is typically mounted to the body of the microscope and suspends a wide-angle lens below the microscope objective, in close proximity to the corneal surface. Though the viewing attachment is not intended to hold the lens in contact with the cornea, the inventors of the present device realized that this could be an effective method of positioning and retaining a lens that would contact the cornea.

The assignee of the present invention (OCULUS GmbH) manufactures wide-angle viewing attachments and adapters to mount to a variety of operating microscopes. One embodiment of the present invention employs a wide-angle viewing attachment and adapter in a method for attaching a novel apparatus (e.g. for positioning a hands-free lens on the cornea) to various operating microscopes.

One type of wide angle-viewing attachment requires sterilization in between uses. In an example, the assignee developed an example of this wide-angle viewing attachment (OCULUS® Binocular Indirect Ophthalmomicroscope or “OCULUS BIOM” herein, and disclosed in U.S. Pat. No. 7,092,152 which is incorporated by reference herein).

Another type of wide angle-viewing attachment is for use as a single-use disposable. In an example, the assignee developed an example of this wide-angle viewing attachment (OCULUS Binocular Indirect Ophthalmomicroscope Ready or “BIOM READY” herein and disclosed in U.S. Pat. No. 9,155,593 which is incorporated by reference herein). In one example, the BIOM READY wide-angle viewing attachment is injection molded and is for use as a single-use disposable.

In one embodiment, the inventors recognized that it would be advantageous to provide an apparatus that attaches a hands-free lens to a wide angle-viewing attachment, such that the apparatus permits the lens to contact the eye without the need to manually hold the lens. The inventors recognized that it would be further advantageous if such an apparatus is designed to accommodate relative movement between the eye and the wide-angle viewing attachment (and/or microscope) along multiple degrees of freedom (e.g. translational and/or rotational). In an example embodiment, the apparatus is made for use with any viewing attachment, such as the OCULUS BIOM or the BIOM READY wide angle viewing attachments. With the BIOM READY wide angle viewing attachment, the apparatus can be used as an all-encompassing disposable system.

Advantageous embodiments of the proposed invention disclose a means to attach a lens (e.g. surgical contact lens) to a wide-angle viewing attachment (e.g. OCULUS BIOM, BIOM READY, etc.) in a method that allows for stable and hands-free positioning of the lens atop the cornea.

In a first set of embodiments, an apparatus is presented for attaching a lens to a microscope with an optical attachment. The apparatus includes the lens with one or more translational degrees of freedom such that the lens is configured to translate along one or more first directions relative to the microscope. The apparatus also includes the lens with one or more rotational degrees of freedom such that the lens is configured to rotate about one or more rotational axes relative to the microscope.

In a second set of embodiments, a system is presented for attaching a lens to a microscope with an optical attachment. The system includes a lens and the optical attachment to attach the lens to the microscope. The optical attachment includes a lens arm and/or the viewing attachment configured to move the lens and the lens arm relative to the microscope.

In a third set of embodiments, a method is presented for using an optical attachment to position a lens relative to a microscope. The method includes securing the lens to a first end of a lens arm of the optical attachment and securing a second end of the lens arm to a positioning device of the optical attachment. The method also includes moving the lens with the positioning device until the lens makes contact with an eye of a patient. The method also includes translating the lens along one or more directions relative to the microscope, based on relative movement between the first end and the second end of the lens arm in the one or more directions such that the lens maintains contact with the eye.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1 is an image that illustrates an example of a system providing a hands-free lens for use during optical surgery, according to an embodiment;

FIG. 2A is an image that illustrates an example of a side view of the lens and lens arm of the system of FIG. 1, according to an embodiment;

FIG. 2B is an image that illustrates an example of a front view of the lens and lens arm of the system of FIG. 1, according to an embodiment;

FIGS. 3A through 3C are images that illustrate an example of the system of FIG. 1 based on relative movement between the lens and microscope in a first direction, according to an embodiment;

FIGS. 4A through 4C are images that illustrate an example of the system of FIG. 1 based on relative movement between the lens and microscope in a second direction, according to an embodiment;

FIGS. 5A through 5C arc images that illustrate an example of the lens and lens arm undergoing the relative movement in the second direction of FIGS. 4A through 4C, according to an embodiment;

FIGS. 6A through 6C are images that illustrate an example of the lens and lens arm of the system of FIG. 1 undergoing relative movement in a third direction, according to an embodiment;

FIGS. 7A through 7C are images that illustrate an example of the viewing attachment of FIG. 1 being rotated about the optical axis of the microscope, according to an embodiment;

FIGS. 8A and 8B are images that illustrate an example of the system of FIG. 1 based on relative movement between the lens and microscope in a first direction, according to an embodiment;

FIGS. 8C and 8D are images that illustrate an example of the system of FIG. 1 based on relative movement between the lens and microscope in a second direction, according to an embodiment;

FIGS. 8E and 8F are images that illustrate an example of the system of FIG. 1 based on relative movement between the lens and microscope in a second direction, according to an embodiment;

FIG. 9A is an image that illustrates an example of interior anatomy of the human eye and an example of total internal reflection;

FIG. 9B is a ray diagram that illustrates an example of light refraction and total internal refraction at a boundary between materials of different indices;

FIG. 10 is an image that illustrates an example of a gonioscopy lens manually held on the eye of a subject;

FIG. 11 is a flowchart that illustrates an example of a method for providing a hands-free lens for use during optical surgery, according to an embodiment; and

FIGS. 12A through 12M are images that illustrate an example of performing one or more of the steps of the method of FIG. 11, according to an embodiment.

### DETAILED DESCRIPTION

A method and apparatus are described for attaching a lens to a microscope with an optical attachment (e.g. for use during a surgical procedure). In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X , for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of optical devices used to treat or examine a patient (e.g. examine the patient, perform surgery on the patient, etc.). In some embodiments, the invention is described in a context of a system provided including a lens, and an apparatus to position the lens and secure the lens to the body of an ophthalmic operating microscope. In one embodiment, the system is intended to safely position the lens onto the eye in a way that is stable and non-obstructive for the user, avoiding the need to manually hold a lens. In another embodiment, a method is provided for using the microscope with the addition of the system, including its installation. In yet another embodiment, a method is provided for forming the system. For purposes of this description, “optical device” means a device with oculars or a camera and an objective lens through which a medical professional views a region of interest of a patient, for diagnostic or therapeutic purposes. In one embodiment, the optical device is an operating microscope (e.g. ophthalmic operating microscope).

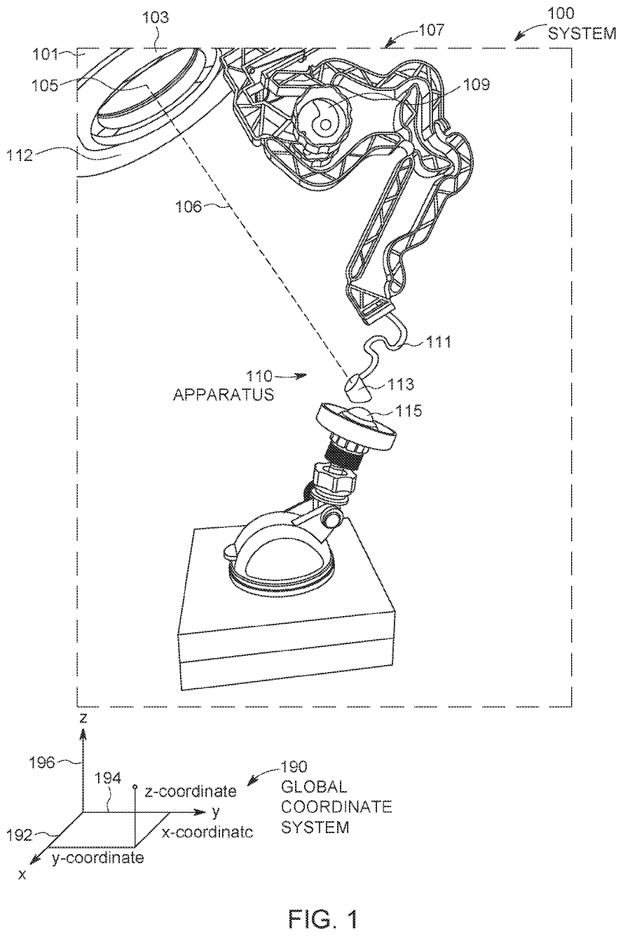

FIG. 1 is an image that illustrates an example of a system 100 providing a hands-free lens for use during optical surgery, according to an embodiment. In an embodiment, the system 100 includes a microscope 101 with an objective lens 103 that defines an objective optical axis 106. In an embodiment, the system 100 also includes an adapter plate 105 and a sterility disc 112 that covers the adapter plate 105 (e.g. to provide a sterile barrier for the microscope 101 and the adapter plate 105). In other embodiments, the system 100 excludes the microscope 101 and the adapter plate 105.

In an embodiment, the system 100 includes an optical attachment to attach a lens 113 to a microscope 101. For purposes of this description, “optical attachment” means one or more components that are used to independently or collectively position the lens 113 at a desired position, relative to the microscope 101. In one embodiment, the optical attachment includes a wide angle viewing attachment 107 (e.g. single use disposable). In an example embodiment, the wide angle viewing attachment 107 is the BIOM READY. In an example embodiment, a first end of the viewing attachment 107 is secured to the adapter plate 105 and a second end opposite to the first end is secured to a lens arm 111 that positions the lens 113 (e.g. on an eye 115). In some embodiments, the optical attachment also includes the lens arm 111. In one embodiment, the viewing attachment 107 has a knob 109 that can be adjusted (e.g. rotated) to vary a separation between the second end (e.g. the lens arm 111 and lens 113) and the microscope objective lens 103 (e.g. along the optical axis 106). In one embodiment, the system 100 includes the lens 113 (e.g. gonioscopy lens) and/or the optical attachment (e.g. the lens arm 111 and the viewing attachment 107) that is configured to move the lens 113 and the lens arm 111 relative to the microscope 101. In some embodiments, the lens 113 is a non-prismatic lens, a plano-concave lens, a mirrored lens, a double mirrored lens, a bioconcave lens or a combination thereof.

In one embodiment, the lens arm 111 and the lens 113 define an apparatus 110 that is used to position or couple the lens 113 to the microscope 101 such that the lens 113 has one or more degrees of freedom to accommodate relative movement between the eye 115 and the microscope 101 (e.g. one or more translational degrees of freedom to accommodate relative translational movement between the eye 115 and the microscope 101 and/or one or more rotational degrees of freedom to accommodate relative rotation between the eye 115 and the microscope 101). In the illustrated embodiment of FIG. 1, the lens 113 is a gonioscopy lens and sits on a model eye 115. However, in other embodiments where an actual patient is present, the lens arm 111 would similarly position the lens 113 on the eye of a patient (e.g. to gently rest on the cornea).

In an embodiment, the viewing attachment 107 (e.g. BIOM READY) is formed in such a way that the lens arm 111 and gonioscopy lens 113 are movable, essentially without resistance, in a direction towards the microscope objective (e.g. along the optical axis 106). In an example embodiment, where the viewing attachment 107 is the BIOM READY, the mechanics of the viewing attachment 107 is disclosed in U.S. Pat. No. 9,155,593, which is incorporated by reference herein. The inventors of the present invention recognized that this feature protects the eye from injury caused by the lens 113 during movement of the patient or movement of the microscope 101.

In an embodiment, a global coordinate system 190 is depicted in FIG. 1. The global coordinate system 190 is merely one example of a coordinate system that can be used to describe the directions of translational and/or rotational degrees of freedom of the apparatus 110 and thus other coordinate systems can be similarly employed. In this embodiment, the coordinate system 190 includes three-dimensional cartesian axes including an x-axis 192, a y-axis 194 and a z-axis 196. The z-axis 196 is defined as parallel to a direction of gravitational acceleration. The y-axis 194 is defined such that the lens arm 111 and lens 113 arc positioned within a y-z plane 195 defined by the y-axis 194 and z-axis 196 (e.g. when the lens arm 111 is in a neutral position, prior to relative translational and/or rotational movement between the lens 113 and microscope 101). The x-axis 192 is defined such that an x-z plane 197 defined by the x-axis 192 and z-axis 196 is orthogonal to the y-z plane 195.

In one example embodiment, the lens arm 111 is configured to accommodate relative translational movement between the lens 113 and microscope in any direction within the y-z plane 195, any direction within the x-z plane 197 and/or any direction within an x-y plane defined by the x-axis 192 and y-axis 194. In another example embodiment, the lens arm 111 is configured to accommodate relative rotational movement between the lens 113 and microscope 103 along multiple rotational axes including axes that are parallel to the x-axis 192, the y-axis 194 and the z-axis 196. For purposes of this description, “accommodate” means that one or more conditions of the system 100 are maintained after the relative translational movement and/or relative rotational movement. For example, one of these conditions is that the lens 113 remains concentric with and/or in spherical contact with the eye 115. In another example, one of these conditions is that an optical axis of the lens 113 remains aligned with, or within an angular threshold of, the optical axis 106 of the microscope objective lens 103.

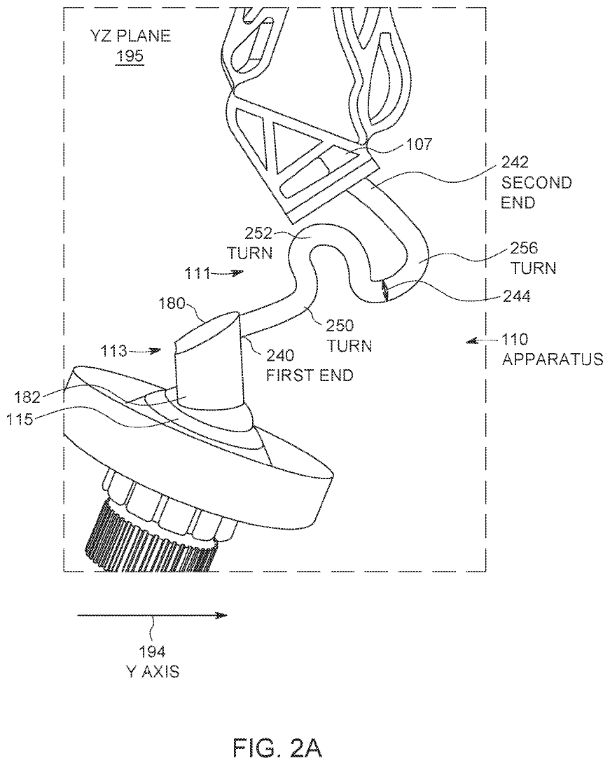

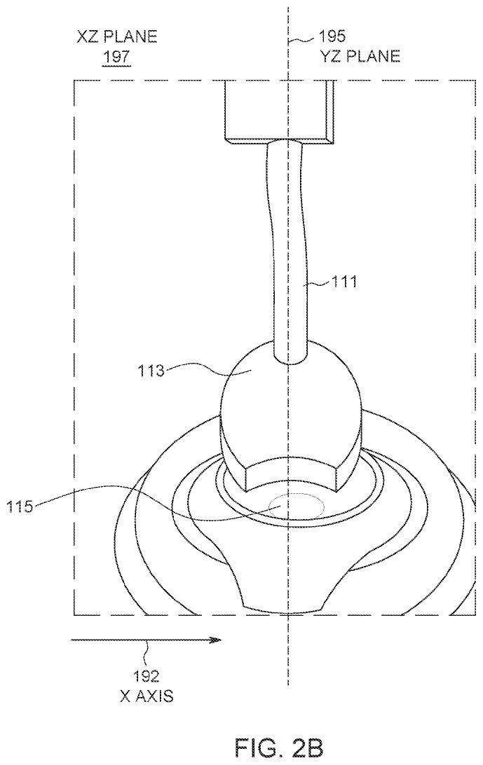

FIG. 2A is an image that illustrates an example of a side view of the lens 113 and lens arm 111 of the system 100 of FIG. 1, according to an embodiment. In an embodiment, the side view of FIG. 2A is taken along the y-z plane 195. FIG. 2B is an image that illustrates an example of a front view of the lens 113 and lens arm 111 of the system of FIG. 1, according to an embodiment. In an embodiment, the front view of FIG. 2B is taken along the x-z plane 197. In an embodiment, the lens 113 features one or more translation degrees of freedom such that the lens 113 is configured to translate along a first direction (e.g. along the objective optical axis 106, along any direction within the y-z plane 195, along any direction within the x-z plane 197, etc.) relative to the microscope 101. In an example embodiment, features of the lens 113 allow the lens 113 to safely be positioned on the cornea of the eye 115 (e.g. filleted edges and a biocompatible material minimize the risk of corneal injury or irritation). The inventors of the present invention recognized that these translational degrees of freedom allow axial positioning of the lens 113 (e.g. along the objective optical axis 106 or any direction within the y-z plane 195) as well as lateral positioning of the lens (e.g. along a direction within the x-z plane 197 or x-y plane orthogonal to the y-z plane 195) independent from the microscope 101 focus position.

In an embodiment, as shown in FIG. 2A, the apparatus 110 includes the lens arm 111 with a first end 240 attached to the lens 113 and a second end 242 that is attached to the optical attachment 107. In one embodiment, the first end 240 is fixedly attached to the lens 113 with any means appreciated by one of ordinary skill in the art (e.g. using UV curable adhesive, Acrylic adhesive or potentially a mechanical attachment such as a press fit, etc.) and the second end 242 is fixedly attached to the optical attachment 107 (e.g. using mechanical attachments such as a press fit/interference fit or a snap clip structure). In one embodiment, the apparatus 110 is configured such that the first end 240 is configured to move relative to the second end 242 (e.g. remains fixed to the positioning device 107) to accommodate one or more translational degrees of freedom and/or one or more rotational degrees of freedom of the lens 115 relative to the microscope 101.

In an embodiment, the lens arm 111 has one or more parameters that are selected in a design phase of the lens arm 111. In one embodiment, this parameter includes a shape of the lens arm 111. In one example embodiment, the shape of the lens arm 111 is selected to include an arcuate or curved surface. In one example embodiment, this curved or arcuate surface includes one or more turns 250, 252, 256 between the first and second ends 240, 242. For purposes of this description, “turn” means a segment of the lens arm 111 between two adjacent linear segments where the lens arm 111 has a radius of curvature. Although FIG. 2A depicts the lens arm 111 having three turns, this is merely one example embodiment and in other embodiments, less or more than three turns are provided. In one example embodiment, a radius of curvature (R) of the turns 250, 252, 256 are selected within a range from about 1 millimeter (mm) to about 40 mm. In an example embodiment, this disclosed range encompasses each of the turns 250, 252, 256 and thus there may be variation in the radius of curvature between the turns 250, 252, 256 (e.g. the radius of curvature of turn 252 is greater than the radius of curvature of turn 250).

In an embodiment, the lens arm 111 is sized using one or more dimensions 244. In an example embodiment, the value of the one or more dimensions 244 is a value of a width or thickness of the lens arm 111 between the first end 240 and the second end 242. In an example embodiment, the value of the width or thickness of the lens arm 111 is in a range from about 1 mm to about 5 mm. In some embodiments, the value of the dimension 244 (e.g. width or thickness) is fixed between the first and second ends 240, 242. In other embodiments, the value of the dimension 244 is varied between the first and second ends 240, 242.

In an embodiment, the lens arm 111 is made from a material having certain properties. In an example embodiment, the lens arm 111 is made from a material having elastic properties such as Thermoplastic Elastomer (TPE) or Thermoset Elastomer (e.g. Silicone, Urethane, vulcanized rubber, etc.). In another example embodiment, the material of the lens arm 111 is selected such that it has certain characteristics, such as an Elongation of about 300% or in a range from about 100% to about 1000% and/or Shore Hardness of about 55 A (Shore A scale) or in a range from about 30 A to about 80 A.

In an embodiment, the lens arm 111 is formed using one or more processes. In an example embodiment, these processes include injection molding, compression molding, casting, 3D printing, or any other similar process appreciated by one of ordinary skill in the art.

In an embodiment, one or more parameter values of the lens arm 111 previously discussed are selected during a design phase of the lens arm 111 to fulfill certain criteria in terms of the relative movement between the eye 115 and the microscope 101. For example, one or more of these parameter values are selected such that upon relative movement between the eye 115 and the microscope 101, the lens arm 111 is configured to rotate the lens 113 on the eye 115 about one or more rotational axes and/or translate the lens 113 and eye 115 relative to the microscope 101 such that the lens 113 remains in concentric contact and/or spherical contact with the eye 115 and the optical axis of the lens 113 remains aligned with (or within an angular threshold) of the optical axis 106 of the microscope objective lens 103

FIG. 2B is an image that illustrates an example of a front view of the lens 113 and lens arm 111 of the system 100 of FIG. 1, according to an embodiment. The front view of FIG. 2B is taken along the x-z plane 197. In one embodiment, FIG. 2B depicts a neutral position of the lens 113 and lens arm 111 which are aligned in the y-z plane 195 (see dotted y-z plane 195 in FIG. 2B) before any translation of the eye 115 relative to the microscope 101 or rotation of the lens 113 relative to the eye 115.

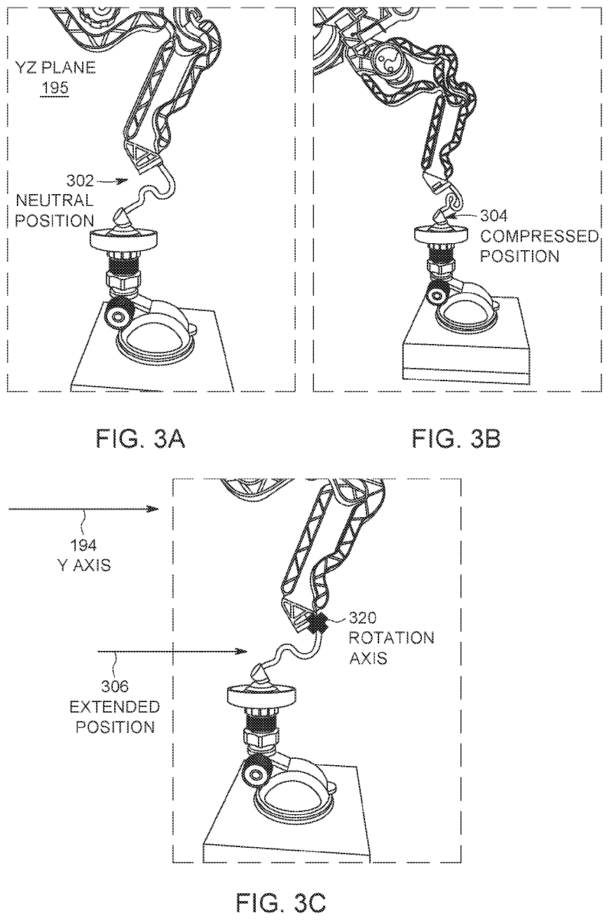

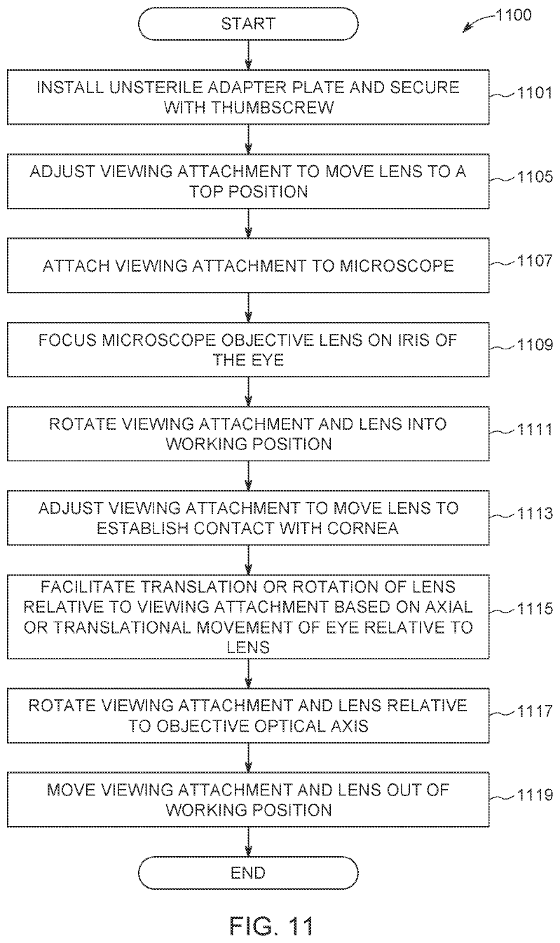

FIGS. 3A through 3C are images that illustrate an example of the system 100 of FIG. 1 based on relative movement between the lens 113 and microscope 101 in a first direction, according to an embodiment. In one embodiment, the first direction is the y-axis 194. As discussed herein, in some embodiments the lens arm 111 has one or more characteristics of a spring (e.g. neutral position, compressed position, extended position, etc.). In an embodiment, FIG. 3A depicts the lens arm 111 in a neutral position 302, which is defined as a position of the lens 113 relative to the microscope 101 prior to any translation of the lens 113 relative to the microscope 101 along the y-axis 194. In an example embodiment, the neutral position 302 is also defined as a position of the lens 113 relative to the microscope 101 after one or more initial setup steps of the system 100 (e.g. see step 1113 of FIG. 11) prior to the medical professional using the system 100 to view the interior of the eye 115.

In an embodiment, FIG. 3B depicts the lens arm 111 in a compressed position 304. In one embodiment, the compressed position 304 of the lens arm 111 results from relative movement between the eye 115 and microscope 101 along the y-axis 194 (e.g. to the right while viewing FIG. 3B). In one example embodiment, this relative movement is translation of the eye 115 relative to the microscope 101 along the y-axis 194 (e.g. to the right while viewing FIG. 3B). This translational movement may be due to shifting of the patient during the medical procedure. In another example embodiment, this relative movement is translation of the microscope 101 relative to the eye 115 along the y-axis 194 (e.g. to the left while viewing FIG. 3B). This translational movement may be due to manual movement of the microscope 101 by the medical professional within the x-y plane (e.g. using a foot pedal apparatus to control movement of the microscope 101 in the x-y plane). In an example embodiment, the compressed position 304 can accommodate lateral movement along the y-axis 194 up to about 30 mm or in a range from about 20 mm to about 40 mm.

In an embodiment, FIG. 3C depicts the lens arm 111 in an extended position 306. In one embodiment, the extended position 306 of the lens arm 111 results from relative movement between the eye 115 and microscope 101 along the y-axis 194 (e.g. to the left while viewing FIG. 3C). In one example embodiment, this relative movement is translation of the eye 115 relative to the microscope 101 along the y-axis 194 (e.g. to the left while viewing FIG. 3C). This translational movement may be due to shifting of the patient during the medical procedure. In another example embodiment, this relative movement is translation of the microscope 101 relative to the eye 115 along the y-axis 194 (e.g. to the right while viewing FIG. 3C). This translational movement may be due to manual movement of the microscope 101 by the medical professional within the x-y plane (e.g. using a foot pedal apparatus to control movement of the microscope 101 in the x-y plane).

In an embodiment, FIG. 3C depicts a rotational axis 320 about which the lens arm 111 is configured to rotate within the y-z plane 195. In one example embodiment, the first end 240 is configured to rotate with respect to the second end 242 about the rotational axis 320. In some example embodiments, this rotation accommodates rotation of the lens 113 with respect to the eye 115 in order to ensure that certain conditions are maintained during the relative translation discussed with respect to FIGS. 3A-3C in the yz-plane 195 (e.g. that the lens 113 remains in concentric and/or spherical contact with the eye 115 and/or the optical axis of the lens 113 remains aligned with or within an angular threshold of the microscope optical axis 106 during the relative translation discussed with respect to FIGS. 3A-3C. In other embodiments, the lens arm 111 is configured to rotate about other rotational axes than the rotational axis 320 in order to accommodate relative translation between the eye 115 and microscope 101 in other directions.

In an embodiment, not only is the first end 240 of the lens arm 111 configured to accommodate the relative translation between the eye 115 and microscope along the y-axis 194, but is further configured to simultaneously accommodate rotation of the lens 113 with respect to the eye 115 during this relative translation. The inventors of the present invention recognized that this advantageously ensures that the lens 113 remains concentric with and/or in spherical contact with the eye 115 and/or the optical axis of the lens 113 remains aligned with (or within an angular range of) the microscope optical axis 106, as the lens 113 is accommodating the translational movement between the eye 115 and microscope 101.

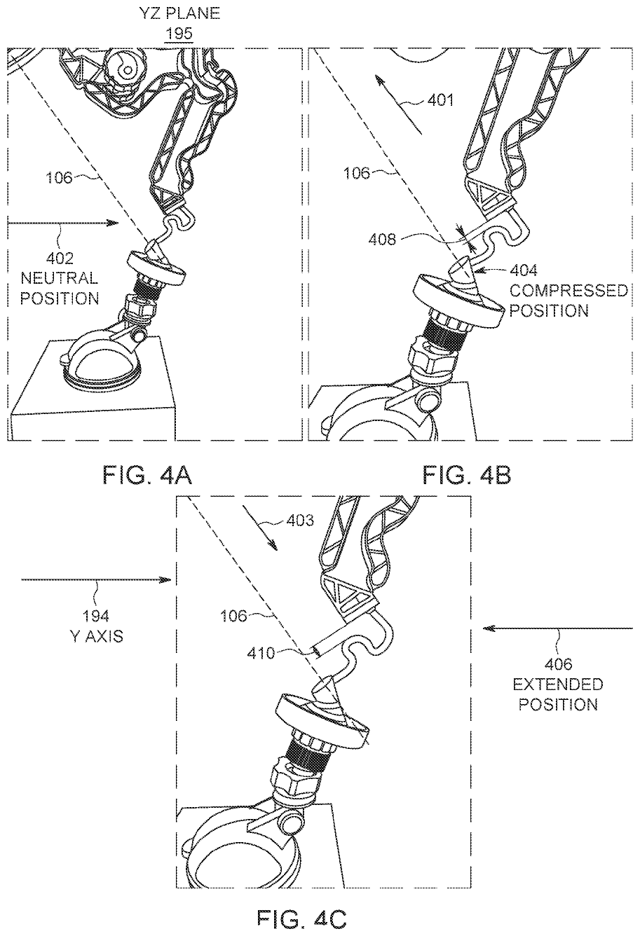

Although FIGS. 3A through 3C show the lens arm 111 accommodating relative translation between the eye 115 and microscope 101 in one direction (e.g. along the y-axis 194 and within the y-z plane 195), in other embodiments the lens arm 111 accommodates relative translation in any direction (e.g. any direction within the y-z plane 195). FIGS. 4A-4C depicts an example embodiment of the lens arm 111 accommodating such relative translation in a different direction within the y-z plane 195 than the accommodation of FIGS. 3A-3C.

FIGS. 4A through 4C are images that illustrate an example of the system 100 of FIG. 1 based on relative movement between the lens 113 and microscope 101 in a second direction, according to an embodiment. In one embodiment, the second direction is the optical axis 106 of the microscope 101. In an embodiment, FIG. 4A depicts the lens arm 111 in a neutral position 402, which is defined as a position of the lens 113 relative to the microscope 101 prior to any translation of the lens 113 relative to the microscope 101 along the optical axis 106. In an example embodiment, the neutral position 402 is also defined as a position of the lens 113 relative to the microscope 101 after one or more initial setup steps of the system 100 (e.g. see step 1113 of FIG. 11) prior to the medical professional using the system 100 to view the interior of the eye 115.

In an embodiment, FIG. 4B depicts the lens arm 111 in a compressed position 404. In one embodiment, the compressed position 404 of the lens arm 111 results from relative movement 401 between the eye 115 and microscope 101 along the optical axis 106 (e.g. towards the objective lens 103). As shown in FIG. 4B, due to this relative movement, a separation between the lens arm 111 and the positioning device 107 is reduced from an initial value (e.g. about 5 mm) to a compressed separation 408 (e.g. less than about 1 mm). In one example embodiment, this relative movement is translation of the eye 115 relative to the microscope 101 along the optical axis 106 (e.g. toward the objective lens 103). This translational movement may be due to shifting of the patient during the medical procedure. In another example embodiment, this relative movement is translation of the microscope 101 relative to the eye 115 along the optical axis 106 (e.g. toward the eye 115). This translational movement may be due to manual movement of the microscope 101 by the medical professional along the optical axis 106 (e.g. using the microscope focus or adjusting the knob 109 on the positioning device 107). In an example embodiment, the compressed position 404 can accommodate movement along the optical axis 106 up to about 20 mm or in a range from about 15 mm to about 25 mm.

In an embodiment, FIG. 4C depicts the lens arm 111 in an extended position 406. In one embodiment, the extended position 406 of the lens arm 111 results from relative movement between the eye 115 and microscope 101 along the optical axis 106 (e.g. away from the optical lens 103). As shown in FIG. 4C, due to this relative movement, a separation between the lens arm 111 and the positioning device 107 is increased from an initial value (e.g. about 5 mm) to an extended separation 410 (e.g. about 15 mm) In one example embodiment, this relative movement is translation of the eye 115 relative to the microscope 101 along the optical axis 106 (e.g. away from the optical lens 103). This translational movement may be due to shifting of the patient during the medical procedure. In another example embodiment, this relative movement is translation of the microscope 101 relative to the eye 115 along the optical axis 106 (e.g. away from the eye 115). This translational movement may be due to manual movement of the microscope 101 by the medical professional along the optical axis 106 (e.g. using a focusing knob or the positioning device 107).

Although FIGS. 4A through 4C show the lens arm 111 accommodating relative translation between the eye 115 and microscope 101 in one direction (e.g. along the optical axis 106 in the y-z plane 195), in other embodiments the lens arm 111 accommodates relative translation in any direction (e.g. any direction within the y-z plane 195).

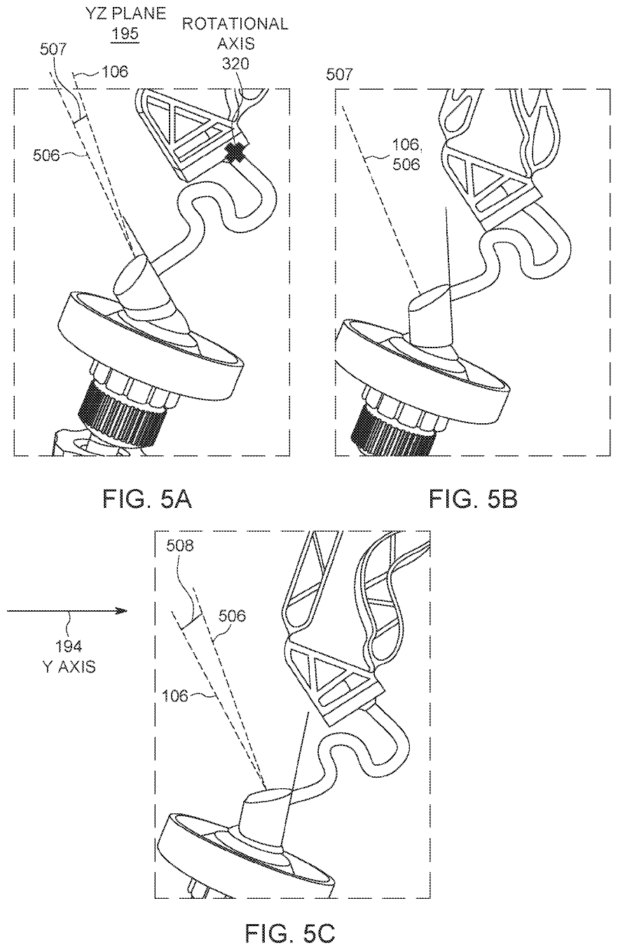

In an embodiment, not only is the first end 240 of the lens arm 111 configured to accommodate the relative translation between the eye 115 and microscope along the optical axis 106 in FIGS. 4A-4C, but is further configured to simultaneously accommodate rotation of the lens 113 with respect to the eye 115 during this relative translation. The inventors of the present invention recognized that this advantageously ensures that the lens 113 remains concentric with and/or in spherical contact with the eye 115 and/or the optical axis of the lens 113 remains aligned with (or within an angular range of) the microscope optical axis 106, as the lens 113 is accommodating the translational movement between the eye 115 and microscope 101 along the optical axis 106. This rotational accommodation of the lens 113 by the lens arm 111 is discussed below with respect to FIGS. 5A-5C.

FIGS. 5A through 5C are images that illustrate an example of the lens 113 and lens arm 111 undergoing the relative movement in the second direction of FIGS. 4A through 4C, according to an embodiment. In one embodiment, FIG. 5B shows a view of the lens 113 and lens arm 111 in the neutral position 402 (FIG. 4A) prior to relative translation of the eye 115 relative to the microscope 101 along the optical axis 106. As shown in FIG. 5B, in this embodiment, an optical axis 506 of the lens 113 is aligned with the optical axis 106 of the microscope 101 in the neutral position 402.

In an embodiment, FIG. 5A depicts a view of the lens 113 and lens arm 111 in the compressed position 402 (FIG. 4B) after relative translation of the eye 115 relative to the microscope 101 along the optical axis 106 (e.g. away from the objective lens 103). In this embodiment, FIG. 5A depicts that the optical axis 506 of the lens 113 has tilted by an angle 507 relative to the optical axis 506 due to this relative translation. In an example embodiment, the angle 507 is less than an angular threshold (e.g. about 30 degrees) or is zero degrees (e.g. in which case the optical axis 506 remains aligned with the optical axis 106).

In an embodiment, FIG. 5C depicts a view of the lens 113 and lens arm 111 in the extended position 404 (FIG. 4C) after relative translation of the eye 115 relative to the microscope 101 along the optical axis 106 (e.g. toward the objective lens 103). In this embodiment, FIG. 5C depicts that the optical axis 506 of the lens 113 has tilted by an angle 508 relative to the optical axis 506 due to this relative translation. In an example embodiment, the angle 508 is less than the angular threshold previously discussed.

In an embodiment, during the relative translation between the eye 115 and microscope 101 along the optical axis 106 discussed above with respect to FIGS. 5A and 5C, the lens arm 111 is configured to rotate about the rotational axis 320 (FIG. 5A) such that the lens 113 rotates on the eye 115 in order to either maintain alignment between the optical axis 506 of the lens 113 and the optical axis 106 of the microscope or at least maintain these optical axes 106, 506 within a maximum angular threshold. The inventor of the present invention recognized that this advantageously ensures that the medical professional can continue to view the interior regions of the eye 115 (e.g. anterior chamber) despite this relative translation along the optical axis 106 or at least can continue to do so with minimal readjustment of the microscope 101.

Although the embodiment of FIGS. 5A through 5C discuss relative movement (e.g. due to eye movement, focusing of microscope or use of the knob 109) between the eye 115 and microscope along the optical axis 106, the embodiments of the present invention are not limited to accommodation of relative movement along the optical axis 106. As previously discussed, the apparatus 100 is also configured to accommodate relative movement in any other direction within the y-z plane 195 (e.g. along the y-axis 194, as discussed with respect to FIGS. 3A through 3C). In an example embodiment, the accommodation of the relative movement along the y-axis 194 in FIGS. 3A through 3C show elongation of the lens arm 111 along the y-axis 194 and prevents the rotation about the rotational axis 320 described in FIGS. 5A through 5C. In the embodiments of the present invention, rotational movements of the lens 113 are a result of the relative translation between the eye 115 and the microscope 101 in order to fulfil the boundary condition of concentric contact between the lens 113 and the eye 115 and/or maintaining alignment between the lens optical axis 506 and the microscope optical axis 106. Thus, in these embodiments, the rotation and translation of the lens 113 are not independent of each other. However, in an embodiment, the degree to which a certain translation results in a rotation of the lens 113 can be influenced by the design and material of the lens arm 111. In an example embodiment, where the lens arm 111 is a straight lens arm (e.g., without the turns 250, 252, 256), this would result in a very pronounced rotation (e.g. about the rotational axis 320) for minor translation along the y-axis 194. In another example embodiment, where the lens arm 111 includes the turns 250, 252, 256, this rotational behavior is drastically reduced. Thus, during the design phase of the lens arm 111, one or more parameters of the design (e.g. whether or not turns are provided, how many turns are provided, the radius of curvature of each turn, etc.) is selected so that the lens arm 111 is configured to undergo the necessary amount of rotation during a translation in any direction, so that these boundary conditions are maintained (e.g. that concentric contact is maintained between the eye 115 and lens 113).

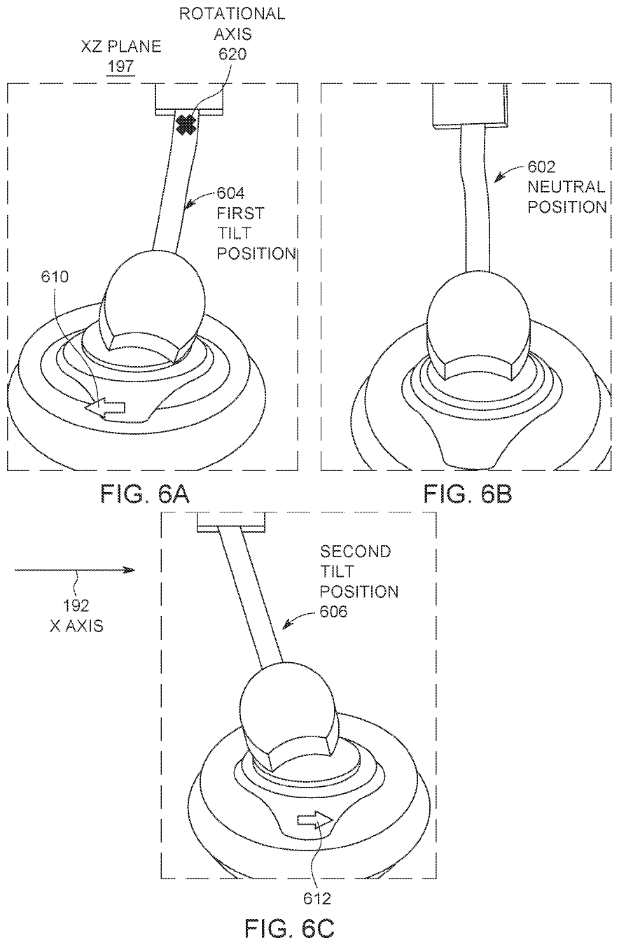

FIGS. 6A through 6C are images that illustrate an example of the lens 113 and lens arm 111 of the system 100 of FIG. 1 undergoing relative movement in a third direction, according to an embodiment. In an embodiment, the third direction is along the x-axis 192. In one embodiment, the lens arm 111 accommodates relative movement along the x-direction 192 based on rotation of the lens arm 111 about a rotational axis 620 (FIG. 6A). In one example embodiment, the rotational axis 620 is parallel to the y-axis 194.

In an embodiment, FIG. 6B depicts the apparatus 110 in a neutral position 602 that is similar to the position depicted and described with respect to FIG. 2B. In this neutral position 602 the lens 113 and lens arm 111 are aligned along the y-z plane 195 after one or more steps to setting the initial position of the system 100 prior to using the system 100 to view the interior of the eye 115 (e.g. steps 1101 through 1113 of FIG. 11).

In an embodiment, FIG. 6A depicts a first tilt position 604 of the apparatus 110 based on relative movement 610 along the x-axis 192 between the eye 115 and the microscope 101. In an embodiment, this relative movement 610 is due to lateral movement of the eye 115 within the x-z plane 197 (to the left looking at FIG. 6B) or is due to lateral movement of the microscope 101 within the x-z plane 197 (to the right looking at FIG. 6B). As shown in FIG. 6A, based on this relative lateral movement 610 along the x-axis 192, the lens arm 111 rotates about the rotational axes 620 so that the lens arm 111 and lens 113 can also undergo the lateral movement 610 to ensure the lens 113 remains in contact with the eye 115. Additionally, during this rotation of the lens arm 111 about the rotational axis 620, the lens 113 is configured to rotate about the eye 115 in order to maintain concentric contact and/or spherical contact with the eye 115 and/or so that the optical axis 506 of the lens 113 maintains alignment (or remains within an angular threshold of) the optical axis 106 of the microscope objective lens 103.

In an embodiment, FIG. 6C depicts a second tilt position 606 of the apparatus 110 based on relative movement 612 along the x-axis 192 between the eye 115 and the microscope 101. In an embodiment, this relative movement 612 is due to lateral movement of the eye 115 within the x-z plane 197 (to the right looking at FIG. 6B) or is due to lateral movement of the microscope 101 within the x-z plane 197 (to the left looking at FIG. 6B). As shown in FIG. 6C, based on this relative lateral movement 612 along the x-axis 192, the lens arm 111 rotates about the rotational axes 620 so that the lens arm 111 and lens 113 can also undergo the lateral movement 620 to ensure the lens 113 remains in contact with the eye 115. Additionally, during this rotation of the lens arm 111 about the rotational axis 620, the lens 113 is configured to rotate about the eye 115 in order to maintain concentric contact and/or spherical contact with the eye 115 and/or so that the optical axis 506 of the lens 113 maintains alignment (or remains within an angular threshold of) the optical axis 106 of the microscope objective lens 103.

In one example embodiment, the lens 113 is injection molded optically clear plastic such as Polymethyl methacrylate (PMMA), Polystyrene (PS), or Polycarbonate (PC). In another example embodiment, the lens 113 is machined glass or quartz/silica lens.

In one embodiment, one or more characteristics of the lens 113 enable the lens 113 to freely move within the motion range afforded by the lens arm 111. In an example embodiment, while retaining the lens 113, one or more characteristics of the lens arm 111 allow the lens 113 to pivot back-to-front, pivot side-to-side and/or move in a translational direction (e.g. vertically along the objective optical axis 106 for optimal positioning or any other direction) and/or rotate about a rotational axis (e.g. axis 320, axis 620 or a third axis that is orthogonal to both axes 320, 620). In an example embodiment, the lens arm 111 also possesses features that allow it to interface and be retained by hardware typically used for a non-contact, wide-angle viewing lens for vitreoretinal procedures (e.g. the viewing attachment 107). In an example embodiment, one or more characteristics of the lens arm 111 also ensure alignment of the imaging lens 113 in the optical axis 106 and for positioning at the proper focal distance.

In an example embodiment, the viewing attachment 107 is configured to move the lens 113 into contact with an eye 115 of a patient. In an example embodiment, the viewing attachment 107 includes an interface (e.g. knob 109) for manual adjustment of the position of the lens 113 relative to the microscope 101 (e.g. along the objective optical axis 106).

In yet another example embodiment, the lens 113 is configured to translate relative to the lens arm 111 in the first direction (e.g. along the objective optical axis 106) by a first extent and the lens 113 is configured to translate relative to the viewing attachment 107 by a second extent that is greater than the first extent. In an example embodiment, the first extent is about 15 mm or in a range from about 1 mm to about 25 mm. In another example embodiment, the second extent is about 35 mm or in a range from about 25 mm to about 45 mm.

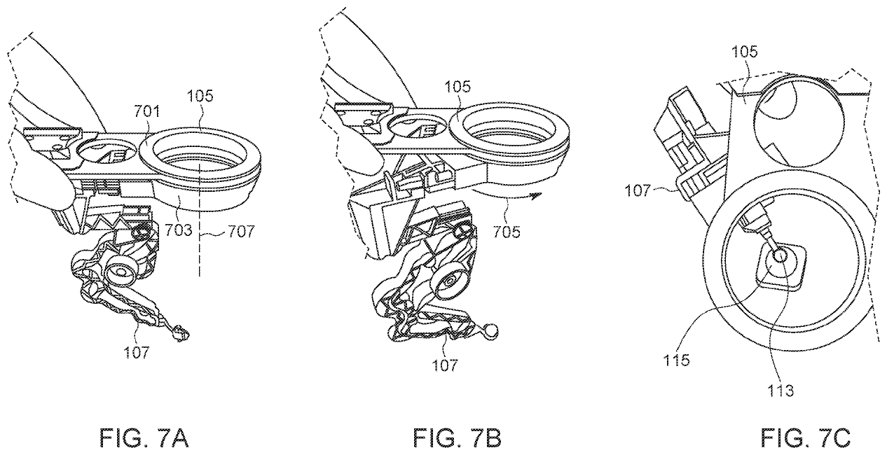

FIGS. 7A through 7C are images that illustrate an example of the viewing attachment 107 of FIG. 1 being rotated about an optical axis 106 of the microscope 101, according to an embodiment. Although the microscope 101 is not depicted in FIGS. 7A through 7C, the adapter plate 105 is depicted with an optical axis 707 that is typically aligned with the optical axis 106 of the microscope 101 when the adapter plate 105 is attached to the microscope 101. Although FIG. 7B depicts that the viewing attachment 107 is rotated in a counterclockwise direction 705, in other embodiments the viewing attachment 107 and lens arm 111 can also be rotated in a clockwise direction (e.g. opposite to the direction 705).

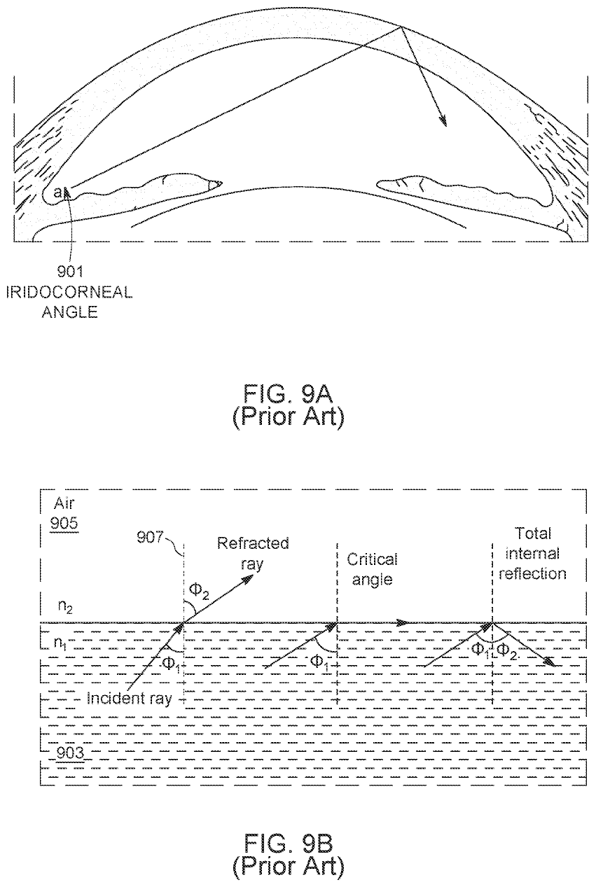

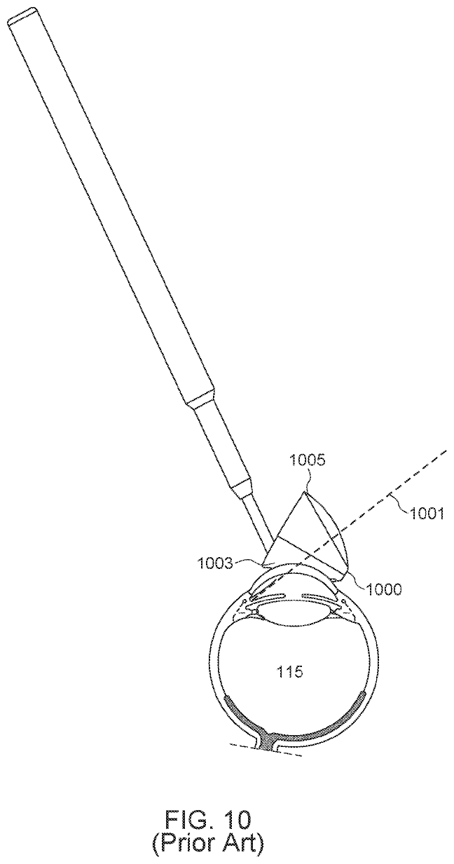

In an embodiment, the rotation of the viewing attachment 107 about the microscope objective axis 106 is provided, enabling the surgeon more field of view for those procedures where surgery is required at different circumferential regions of the eye. In an embodiment, FIGS. 7A through 7C show the adapter plate 105 in two different positions. In this embodiment, the adapter plate 105 is formed in two parts, an upper portion 701 and a lower portion 703 that is configured to pivot (e.g. about the axis 707 that is aligned with the microscope optical axis 106). In an example embodiment, the upper portion 701 is attached to the microscope 101. The upper portion 701 is subsequently locked into place while the lower portion 703 can rotate (e.g. over 360 degrees) about the optical axis 707. In the example embodiment, a method of using of the viewing attachment 107 involves using the rotating ability of the adapter 105 (e.g. up to about +30 degrees in each direction to be able to rotate a gonioscopy lens 113 on the eye 115). This advantageously extends the viewing sector of the iridocorneal angle 901 (FIG. 9A). In FIGS. 7B and 7C, the lower portion 703 of the adapter 105 has been rotated a specific angle (e.g. 30 degrees) counterclockwise, and consequently the gonioscopy lens 113 has been rotated by this specific angle counterclockwise as well.

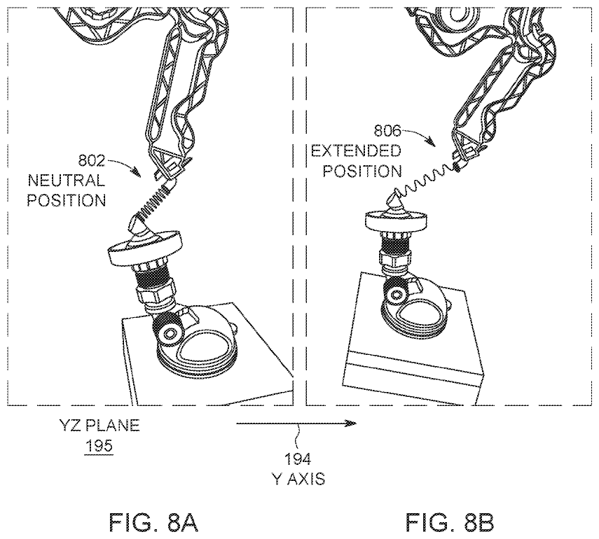

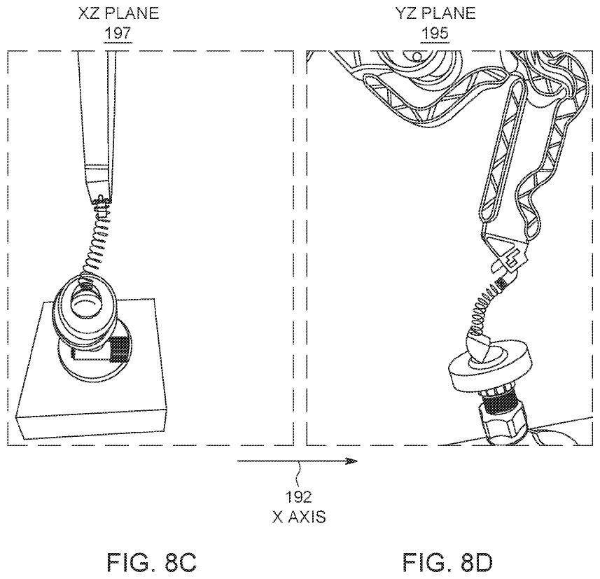

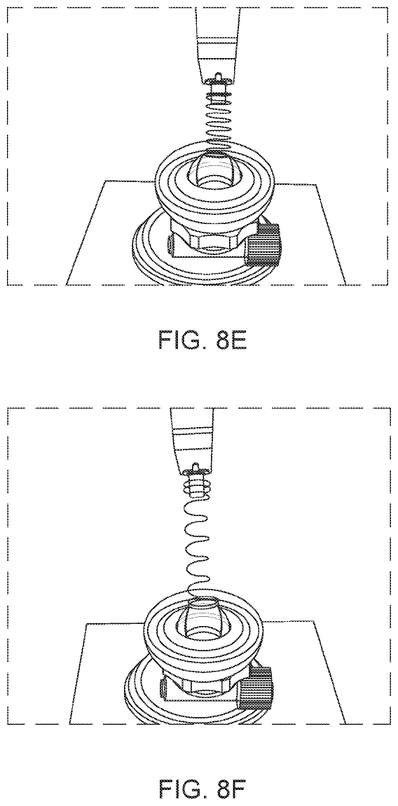

FIGS. 8A and 8B are images that illustrate an example of the system 100 of FIG. 1 based on relative movement between the lens 113 and microscope 101 in a first direction (e.g. along the y-axis 194), according to an embodiment. In an embodiment, the system 100 shown in FIGS. 8A and 8B features a different lens arm design than the lens arm 111. In an example embodiment, as with the lens arm 111, the lens arm of FIGS. 8A and 8B has one or more characteristics of a spring (e.g. neutral position, extended position, etc.). In an example embodiment, FIG. 8A depicts a neutral position 802 that is similar to the neutral position 402 of FIG. 4A for the lens arm 111 design. In another example embodiment, FIG. 8B depicts an extended position 806 that is similar to the extended position 406 of FIG. 4C for the lens arm 111 design. In an embodiment, one or more differences in the design parameters of the lens arm of FIGS. 8A and 8B relative to the lens arm 111 include location of the moment controlling the axis of rotation 320, 620; material selection to adjust the resistive force to relative movement (such as axial displacement 401 and lateral displacement 610) between the patient eye and microscope.

FIGS. 8C and 8D are images that illustrate an example of the system 100 of FIG. 1 based on relative movement between the lens 113 and microscope in a second direction (e.g. along the x-axis 192), according to an embodiment. In an example embodiment, as with the lens arm 111, the lens arm of FIGS. 8C and 8D has one or more characteristics of a spring (e.g. neutral position, extended position, etc.). In an example embodiment, FIG. 8C depicts a first tilt position that is similar to the first tilt position 604 of FIG. 6A for the lens arm 111 design. In another example embodiment, FIG. 8D depicts an extended position that is similar to the extended position of FIG. 5C for the lens arm 111 design.

FIGS. 8E and 8F arc images that illustrate an example of the system 100 of FIG. 1 based on relative movement between the lens 113 and microscope 101 in a second direction, according to an embodiment. In an embodiment, the second direction is along the optical axis 106 of the microscope objective lens 103. In one embodiment, FIG. 8E is similar to the compressed position 404 of FIG. 4B (e.g. after the eye 115 translates towards the lens 103 along the objective axis 106). In another embodiment, FIG. 8F is similar to the extended position 406 of FIG. 4C (e.g. after the eye 115 translates away from the lens 103 along the objective lens 106).

In one embodiment, as shown in FIG. 2A, the lens 113 includes a first surface 182 (e.g. bottom surface) that contacts the eye 115 (e.g. cornea). In an example embodiment, the first surface 182 has a curvature (e.g. concave surface) that is based on a curvature of the cornea so that the first surface 182 remains in contact with and concentric with the cornea (e.g. during the rotation about the axes 320, 620). In an example embodiment, the curvature of the first surface 182 is about equal (e.g. within ±20%) of the curvature of the cornea. In an embodiment, the lens 113 includes a second surface 180 (e.g. top surface). In some embodiments, the lens 113 is a non-prismatic lens and/or a plano-concave lens (e.g. no angle between the axes of the first surface 182 and the second surface 180).

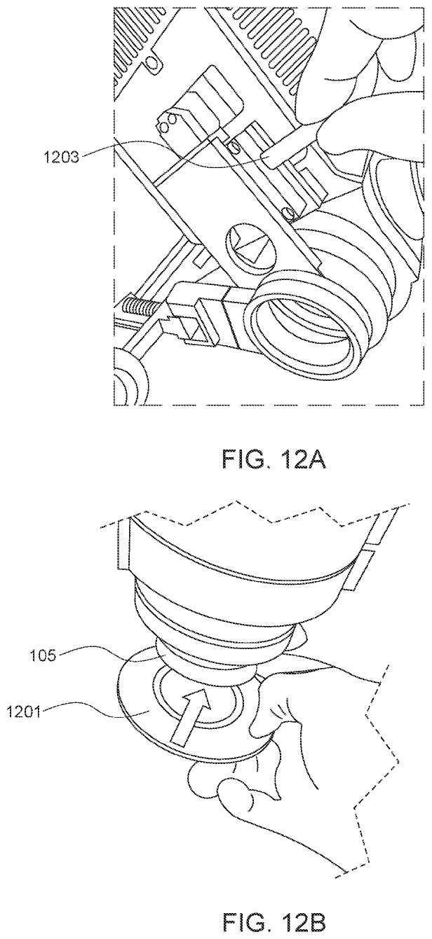

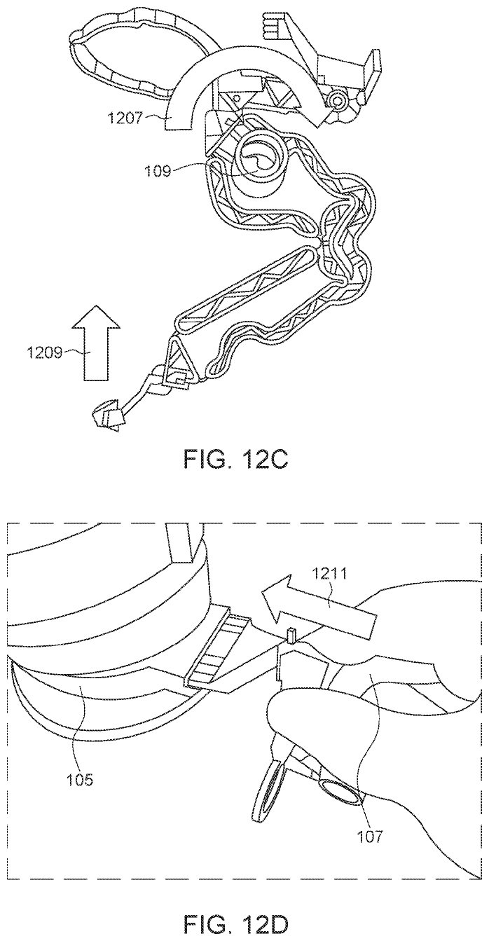

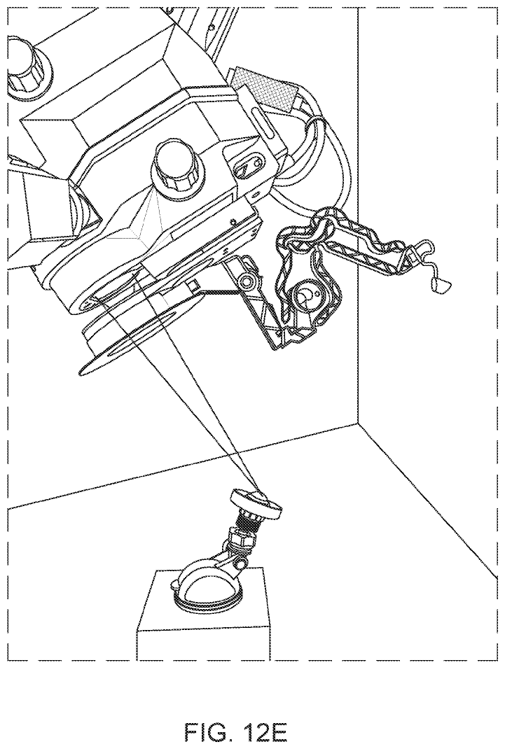

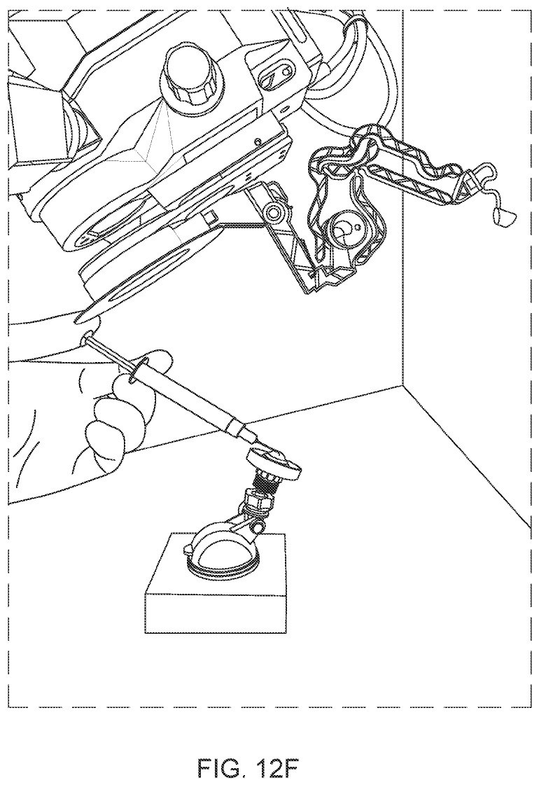

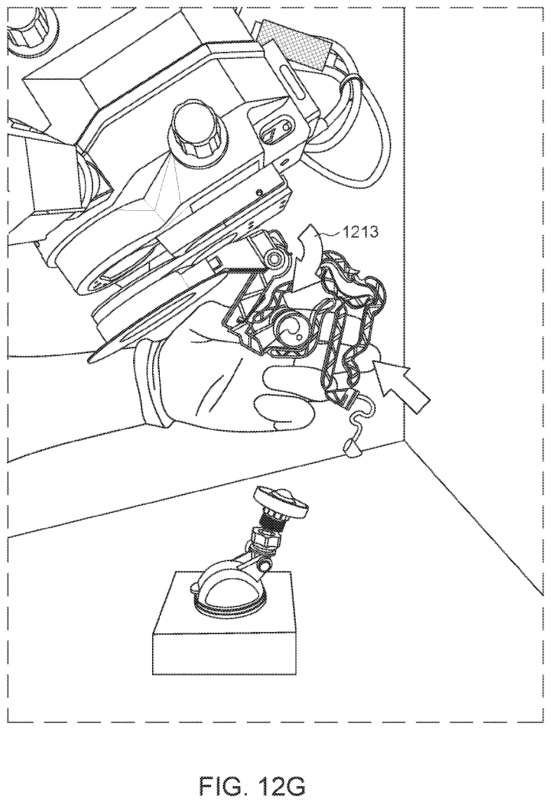

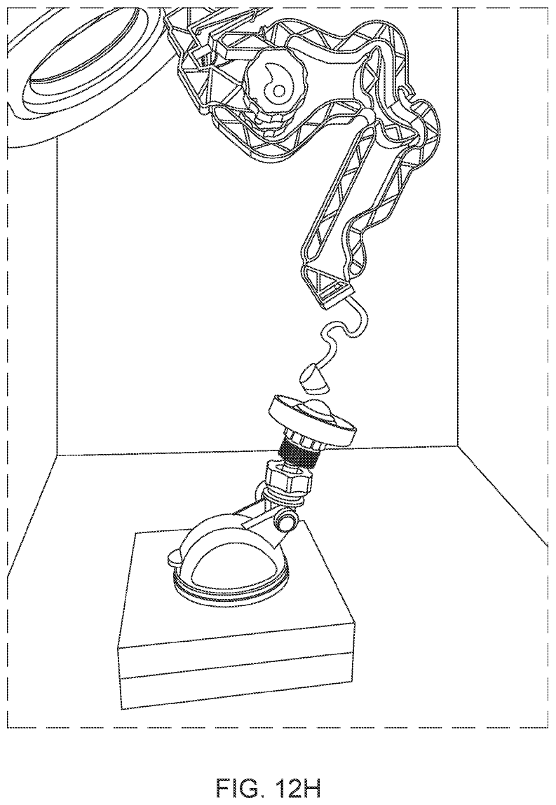

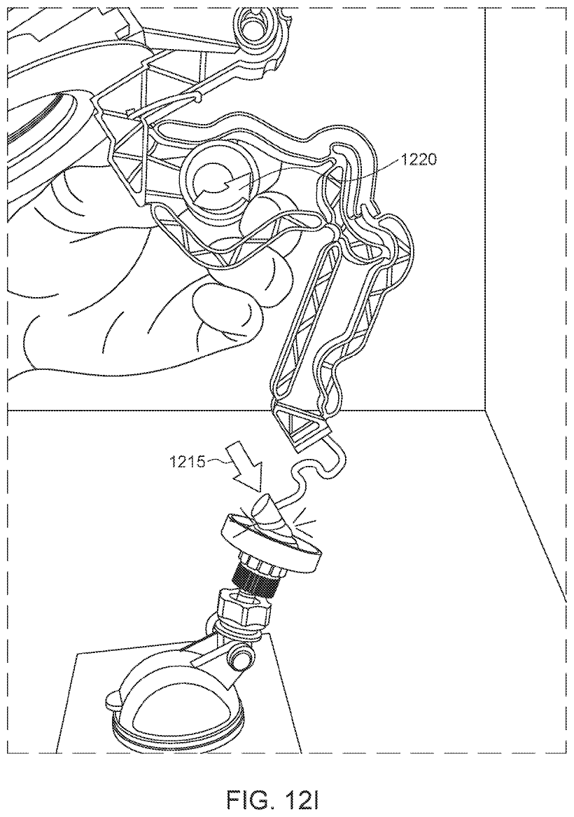

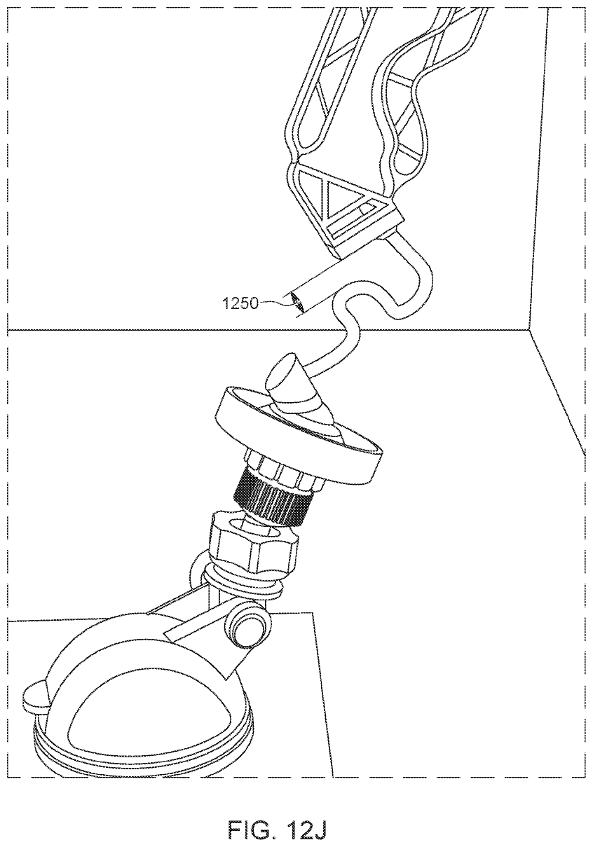

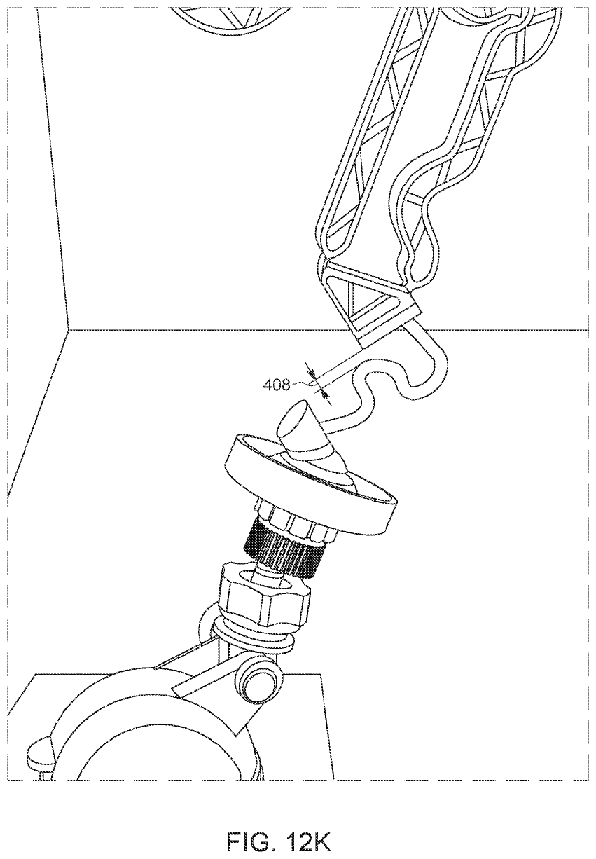

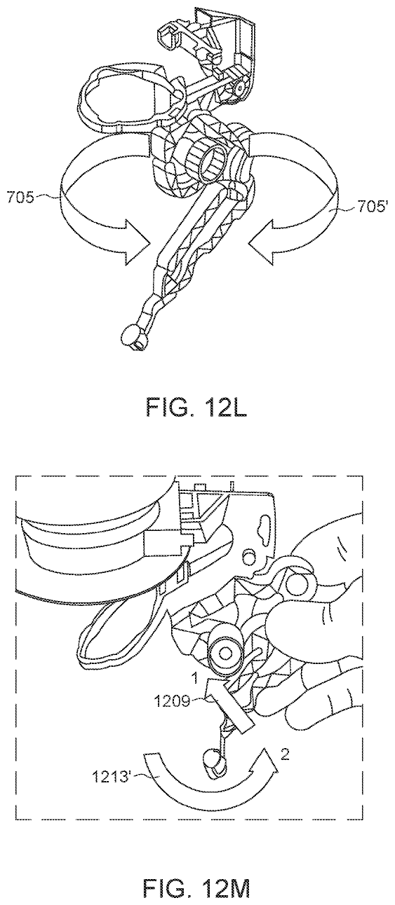

As shown in FIG. 2A, the lens 113 is positioned on the eye 115. In an example embodiment, the ophthalmic operating microscope 101 is tilted by a certain angle (e.g. about 30 degrees or in a range from about 20 degrees to about 50 degrees) from the vertical direction (e.g. z axis 196). In an example embodiment, this tilting of the microscope 101 is performed for surgeons use when performing a procedure involving the iridocorneal angle 901 (FIG. 9A). Thus, for other procedures (e.g. surgeries that do not involve the iridocorneal angle and/or viewing the eye for purposes other than surgery, such as diagnosis) the microscope 101 need not be tilted at this angle. In an embodiment, the first surface 182 is configured to remain in contact with the eye 115 so that light from within the eye 115 passes from the cornea into the lens 113 with minimal refraction (e.g. the difference between the index of refraction of the eye 115 and lens 113 is minimal at the interface between the eye 115 and lens 113). In an example embodiment, to prevent undesired refraction at the interface of the eye 115 and lens 113, solution is applied between the eye 115 (see FIG. 12F) and the lens 113 to reduce the instance of air gaps between the eye 115 and lens 113 (e.g. which would induce unwanted refraction at the eye/air and/or air/lens boundaries). In another example embodiment, the second surface 180 is angled such that incident light from within the lens 113 on the second surface 180 has a minimal incident angle relative to the normal to the second surface 180. The inventors of the present invention recognized that this minimalization of the incident angle on the second surface 180 reduces the likelihood that the incident light on the second surface 180 will undergo TIR and be reflected back into the lens 113 and instead will be transmitted through the second surface 180 and along the objective optical axis 106.

In one embodiment, the lens 113 is configured to be in contact and concentric with an eye 115 of a subject, such that the multiple translational degrees of freedom (e.g. along the first direction, such as along the objective optical axis 106 direction within the yz plane 195; along any second direction within the yz plane 195 such as the y-axis 194; along any third direction within the x-z plane 197 such as the x-axis 192 or any direction within the x-y plane) and the multiple rotational degrees of freedom (e.g. about the first rotation axis 320, the second rotational axis 620 or third rotational axis that is orthogonal to the first and second rotational axes) is to accommodate movement of the eye in the first direction, the second direction or the third direction such that the lens 113 remains in contact and concentric with the eye 115 during any of these directions. As shown in FIGS. 4A through 4C, rotation of the lens 113 about the first rotational axis 320 is provided to maintain contact and concentricity in the case of axial displacement 401 or 403 along the optical axis 106. As shown in FIGS. 6A through 6C, rotation of the lens 113 about the second rotational axis 620 is provided to maintain contact and concentricity in the case of lateral displacement (e.g. movement along the x-direction 192).

In another embodiment, the lens 113 is configured to be in contact and concentric with the eye 115 of the subject such that the second rotational degree of freedom (e.g. about the second rotational axis 620) is configured to accommodate lateral movement of the eye 115 in a lateral direction (e.g. x-axis 192) orthogonal to the first direction (e.g. y-axis 194 or the optical axis 106) such that the lens 113 remains in contact and concentric with the eye 115 during this movement in the lateral direction. In an example embodiment, FIGS. 6A through 6C depicts a lateral displacement 610 or 612 which is accommodated by the rotation about the second rotational axis 620. In an example embodiment, the lateral displacement 610 or 612 is about ±4 mm or in a range from about ±1 mm to about ±15 mm. In yet another example embodiment, the axial displacement 401 or 403 is about ±4 mm or in a range from about ±1 mm to about ±15 mm.

In an embodiment, the rotation of the lens 113 about the second rotational axis 620 is based on the lens 113 and lens arm 111 pivoting about the rotational axis 620 such that the lens 113 pivots on the eye 115 to maintain concentric and/or spherical contact with the eye 115 (FIG. 6A and 6C). In an example embodiment, as shown in FIG. 6A or 6C, as the lens 113 and lens arm 111 pivot about the second rotational axis 802, the lens 113 pivots on the eye 115.

In one embodiment, the first surface 182 (e.g. bottom surface contacting the eye 115) is a concave surface with a curvature that is based on a curvature of the eye such that the first surface 182 is configured to be in contact and concentric with the eye. In another embodiment, the bottom/first surface 182 of the lens 113 is concave with a radius of curvature matching the radius of curvature of the cornea (e.g. about 8 mm or in a range from about 7 mm to about 9 mm) such that the lens 113 (e.g. made of a material with a similar index of refraction to the human cornea) minimizes the refractive power of the cornea. In another example embodiment, the second/top surface 180 of the lens 113 can have various designs each used to visualize a different region or anatomical feature within the eye and/or to control the magnification of the image. In one example embodiment, a second/top surface 180 is convex. In yet another example embodiment, the second/top surface 180 is angled by a certain angle (e.g. about 40 degrees or in a range from about 30 degrees to about 50 degrees). In other embodiments the lens 113 is a prismatic lens for gonioscopy. In still other embodiments, the lens 113 is a plano-concave lens, a bi-concave lens, and/or a convex-concave lens with spherical or aspheric surfaces. In some embodiments the lens 113 has an anti-reflective coating. In still other embodiments, the lens 113 is made of a gamma-stable material.

In an embodiment, a safety feature is provided by the apparatus 110, i.e. to allow intended (focusing) or unintended microscope movement without exerting a force onto the eye which could lead to injury. In an embodiment, the motion range of this safety feature should exceed the focus range necessary to view the eye structures to be examined, as well as exceed most expected unintended microscope movements. In an embodiment, this safety feature is achieved by a provision to allow tilt, rotation, and axial movement of the lens 113 to compensate for minor patient and eye movement, and allow lateral misalignment of the eye relative to the optical axis 106 of the microscope, to ensure continuous contact of the lens-cornea interface. The inventors of the present invention realized that this feature provides a constant, minimal contact force, in order to prevent compression of the anterior chamber during the procedure.

In an embodiment, the second surface 180 is angled at about 50 degrees (or in a range from about 40 degrees to about 60 degrees) relative to the first surface 182 to accommodate a wide range of microscope angles and eye anatomies in the visualization of the iridocorneal angle 901 (FIG. 9A).

FIG. 11 is a flowchart that illustrates an example of a method 1100 for providing a hands-free lens for use during optical surgery, according to an embodiment. Although steps are depicted in FIG. 11 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways. FIGS. 12A through 12M are images that illustrate an example of one or more of the steps of the method 1100 being performed.

In an embodiment, in step 1101 an unsterile adapter plate is installed and secured with a thumbscrew (FIG. 12A). Additionally, in an embodiment a sterility disc 1201 is positioned over the adapter plate 105 (FIG. 12B).

In an embodiment, in step 1105 the lens 113 and lens arm 111 are secured to the viewing attachment. In one embodiment, in step 1105 the lens arm 111 is secured to the viewing attachment 107 using various features. In one embodiment, in step 1105 after the lens 113 and lens arm 111 are secured to the viewing attachment 107, the viewing attachment 107 is adjusted so that the lens 113 is at a top position (e.g. maximum position of a range of movement of the viewing attachment in an upward direction). In an example embodiment, in step 1105 the knob 109 of the viewing attachment 107 is rotated in a first direction 1207 (FIG. 12C) so to move the lens 113 and lens arm 111 in a first direction 1209. In this example embodiment, the knob 109 is rotated in the direction 1207 until the lens 113 is at a top position of the viewing attachment 107. In other embodiments, in step 1105 the viewing attachment is used and the knob is rotated until the lens 113 is at the top position.

In an embodiment, in step 1107 the viewing attachment is attached to the microscope 101. In one embodiment, in step 1107 a portion of the viewing attachment 107 (FIG. 12D) is moved in a direction 1211 such that the portion of the viewing attachment 107 is received in a slot of the adapter plate 105. In an example embodiment, in step 1107 the portion of the viewing attachment 107 is moved into the slot of the adapter plate 105 until the viewing attachment 107 is securely engaged to the adapter plate 105.

In an embodiment, in step 1109 the objective lens 103 of the microscope 101 is focused to the proper distance. In one embodiment, in step 1309 the objective lens 103 is focused on the iris of the eye 115. FIG. 12E depicts an example of step 1109, where the objective lens 103 is focused on the iris of the eye 115.

In some embodiments, after step 1109 gel is placed on the eye 115. FIG. 12F depicts one embodiment where this step is being performed. In these embodiments, the gel is applied to the surface of the eye 115 to prevent and/or minimize total internal reflection (TIR) that occurs at the interface of the eye 115 and lens 113 by eliminating/minimizing any air gaps between the eye 115 and the lens 113.

In an embodiment, in step 1111 the viewing attachment is moved to a working position (e.g. a position where the surgeon can view the eye for purposes of performing one or more surgical procedures and/or can diagnose one or more conditions of the eye). In one embodiment, in step 1111 the viewing attachment 107 (e.g. and the lens 113 and lens arm 111) is rotated in a direction 1213 (FIG. 12G to FIG. 12H) to the working position. In an example embodiment, the working position is a position where the lens 113 is aligned with the objective optical axis 106 of the objective lens 103 (FIG. 12H). As shown in FIG. 12H, in one embodiment, after moving the lens 113 and lens arm 111 in the direction 1209 to the top position (step 1105) the viewing attachment 107 is rotated in the direction 1213 until the lens 113 and lens arm 111 are in the working position.

In an embodiment, in step 1113 the viewing attachment is adjusted to move the lens 113 to establish contact with the eye 115 (e.g. cornea). In one embodiment, in step 1113 the viewing attachment 107 is adjusted by rotating the knob 109 in a second direction 1220 (FIG. 12I) that is opposite to the first direction 1207 of step 1105 (FIG. 12C). In an example embodiment, in step 1113 the viewing attachment is adjusted such that the lens 113 is slowly lowered in a downward direction 1215 (FIG. 12I) that is opposite from the upward direction 1209 in step 1105. In an example embodiment, in step 1113 the adjustment of the viewing attachment is stopped once the lens 113 has made full contact with the cornea. In an example embodiment, in step 1113 once full contact is established between the lens 113 and the cornea, the focus of the microscope objective lens 103 is adjusted to optimize a view of the eye 115 at the iridocorneal angle 901 with the lens 113 (e.g. to correct for change in the optical path length with the lens 113 in place). In an example embodiment, in step 1113 focusing down may result in a slight compression of the flexible lower arm and a temporary increase in the pressure of the eye. In an example embodiment, the method 1100 advantageously ensures that the pressure of the lens 113 on the eye 115 does not exceed a threshold force (e.g. about 1 Newton (N)).

In an embodiment, in step 1113 the viewing attachment is adjusted to move the lens 113 is positioned in the neutral position 402, 502 (FIGS. 4A and 5A) to facilitate axial movement of the lens 113 relative to the lens arm 111 (and viewing attachment). In an example embodiment, in step 1113 the viewing attachment is adjusted until the lens arm 111 is separated by an intermediate separation 1250 from the viewing attachment 107. In an example embodiment, the intermediate separation 1250 is the separation between the lens arm 111 and viewing attachment 107 when the apparatus 110 is in the neutral position 402, 502. In another example embodiment a value of the intermediate separation 1250 is between a value of the compressed separation 408 and extended separation 408 of when the lens arm 111 is respectively in the compressed position 404 and extended position 406. Thus, in step 1113 the lens arm 111 is moved until it is in an intermediate position along the optical axis 106. The inventors of the present invention recognized that this advantageously facilitates axial translation of the lens 113 relative to the microscope 101 in both directions along the optical axis 106 (e.g. the lens 113 moving away from or toward the microscope 101) in the event of axial translation in either direction.

In an embodiment, the objective lens 103 of the microscope 101 is refocused, after the lens 113 is positioned between the eye 115 and the objective lens 103 in step 1113. In one embodiment, as the objective lens 103 is focused, the microscope 101 tends to move slightly towards the eye 115 (e.g. along the optical axis 106). Thus, in this step, the lens arm 111 moves from the neutral position 402 to the compressed position 404 (FIG. 4A to FIG. 4B) which results in the lens arm 111 moved within the compressed separation 408 of the viewing attachment 107 (FIG. 12K). Thus, the inventors recognized that this apparatus 110 advantageously accommodates the initial set up of the system 100 by facilitating axial translation of the microscope 101 relative to the eye 115 along the optical axis 106 of the microscope objective lens 103.

In an embodiment, in step 1115 relative movement between the eye 115 and microscope 101 is facilitated, in one or more translational degrees of freedom and/or one or more rotational degrees of freedom, by adjusting the location of the lens 113 on the eye 115. In an embodiment, the one or more translational degrees of freedom include accommodating relative translation between the eye 115 and microscope 101 in any direction (e.g. along the optical axis 106, along the y-axis 194) in the y-z plane 195; any direction (e.g. along the x-axis 192) in the x-z plane 197 and any direction along the x-y plane. In another embodiment, the one or more rotational degrees of freedom include accommodating relative rotation of the eye 115 about the microscope 101 or rotation of the of the lens 113 over the eye 115 (e.g. around the first rotational axis 320, the second rotational axis 620 or a third rotational axis perpendicular to both axes 320, 620). In one embodiment, in step 1115 relative translational movement between the eye 115 and microscope 101 in any of these directions is facilitated based on translational movement of the lens arm 111 relative to the microscope 101 in the same direction. In another embodiment, in step 1115 concentric and/or spherical contact between the lens 113 and the eye 115 is maintained during this translational movement based on rotational movement of the lens 113 on the eye 115 about one or more of the first rotational axis 320, the second rotational axis 620 or the third rotational axis that is orthogonal to both axes 320, 620.

In an embodiment, in step 1117 the viewing attachment (and lens 113) is rotated relative to the microscope 101. In one embodiment, in step 1117 the viewing attachment 107 is rotated about the optical axis 106 of the microscope objective lens 103. In an example embodiment, the viewing attachment 107 is rotated in a counterclockwise direction 705 (FIG. 12L) or a clockwise direction 705′. In an embodiment, step 1117 is performed to achieve an expanded view of the interior chamber of the eye at an angle (e.g. iridocorneal angle 901). In an example embodiment, in step 1117 the viewing attachment is rotated within an angular range (e.g. about 30 degrees) in the directions 705, 705′. In another example embodiment, during the rotation of step 1117, the lens 113 is not separated from the cornea, and thus provides the surgeon with an expanded view of the eye 115 along the angle (e.g. an expanded view of the anterior chamber of the eye at the iridocorneal angle 901, where the anterior chamber can be viewed at the angle 901 from different orientations, etc.).

In an embodiment, in step 1119 the viewing attachment is moved out of the working position. In one embodiment, step 1119 is performed after the procedure (e.g. eye surgery). In an embodiment, in step 1119 the viewing attachment 107 (and lens 113) are moved out of the working position. In an example embodiment, step 1119 is a reverse of step 1111, where the viewing attachment 107 is adjusted to move the lens 113 in the upward direction 1209 and/or is rotated about a direction 1213′ (FIG. 12M) to move the viewing attachment 107 (and lens 113) out of the working position.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

What is claimed is:

1. An apparatus for attaching a lens to a microscope with an optical attachment, said apparatus comprising at least one of:
the lens with one or more translational degrees of freedom such that the lens is configured to translate along one or more directions relative to the microscope; and
the lens with one or more rotational degrees of freedom such that the lens is configured to rotate about one or more rotational axes relative to the microscope.

2. The apparatus as recited in claim 1, wherein said microscope includes an objective lens that defines an objective optical axis, and wherein the one or more directions include a first direction that is along the objective optical axis.

3. The apparatus as recited in claim 1, wherein said microscope includes an objective lens that defines an objective optical axis, and wherein the one or more directions include a first direction that is angled relative to the objective optical axis.

4. The apparatus as recited in claim 1, wherein the lens has multiple translational degrees of freedom defining multiple directions such that the lens is configured to translate in any of the multiple directions relative to the microscope.

5. The apparatus as recited in claim 1, wherein the optical attachment includes a lens arm and a positioning device to move the lens and the lens arm relative to the microscope;
wherein the apparatus further includes the lens arm with a first end attached to the lens and a second end attached to the positioning device, wherein the first end is configured to move relative to the second end from a first position to a second position to accommodate the translation of the lens in a first direction of the one or more directions.

6. The apparatus as recited in claim 5, wherein an optical axis of the lens is aligned with an optical axis of an objective lens of the microscope when the first end is in the first position and wherein the optical axis of the lens is aligned within an angular threshold of the optical axis of the objective lens when the first end is in the second position.

7. The apparatus as recited in claim 5,
wherein the first end is configured to translate relative to the second end in the first direction to accommodate the translation of the lens in the first direction; and
wherein the first end is configured to rotate relative to the second end about one or more rotational axes such that an optical axis of the lens remains aligned within an angular threshold of an optical axis of an objective lens of the microscope as the first end moves from the first position to the second position.

8. The apparatus as recited in claim 7, wherein the lens and the lens arm are arranged in a first plane when the first end is in the first position and wherein the rotational axis is angled with respect to the first plane.

9. The apparatus as recited in claim 8, wherein the one or more rotational axes comprise:
a first rotational axis that is orthogonal with respect to the first plane;
a second rotational axis that is orthogonal to the first rotational axis; and
a third rotational axis that is orthogonal to the first rotational axis and the second rotational axis.

10. The apparatus as recited in claim 1, wherein the lens is configured to be in contact and concentric with an eye of a subject, such that:
the one or more translational degrees of freedom are to accommodate movement of the eye in a first direction of the one or more directions such that the lens remains in contact and concentric with the eye during said movement in the first direction; and
the one or more rotational degrees of freedom are configured to accommodate lateral movement of the eye in a lateral direction different than the first direction such that the lens remains in contact and concentric with the eye during said movement in the lateral direction.

11. The apparatus as recited in claim 5, wherein the lens arm comprises an arcuate surface between the first end and the second end.

12. The apparatus as recited in claim 11, wherein the arcuate surface defines one or more turns and wherein a radius of curvature of the one or more turns is selected to accommodate at least one of:
translation of the first end relative to the second end in the first direction to accommodate the translation of the lens in the first direction; and
rotation of the first end relative to the second end about one or more rotational axes such that an optical axis of the lens remains aligned within an angular threshold of an optical axis of an objective lens of the microscope as the first end moves from the first position to the second position.

13. The apparatus as recited in claim 5, wherein the lens arm comprises:
an arcuate surface positioned between the first end and the second end and having a value of a radius of curvature in a first range;
a material with a value of an elasticity in a second range; and
a dimension having a value in a third range;
wherein the radius of curvature value in the first range, the elasticity value in the second range and the dimension value in the third range are selected to accommodate:
translation of the first end relative to the second end in the first direction to accommodate the translation of the lens in the first direction; and
rotation of the first end relative to the second end about one or more rotational axes such that an optical axis of the lens remains aligned within an angular threshold of an optical axis of an objective lens of the microscope as the first end moves from the first position to the second position.

14. The apparatus as recited in claim 1, wherein the lens has a first surface configured to be in contact with an eye of a patient and a second surface opposite to the first surface.

15. The apparatus as recited in claim 14, wherein the first surface is a concave surface with a curvature that is based on a curvature of the eye such that the first surface is configured to be in contact and concentric with the eye.

16. The apparatus as recited in claim 14, wherein the first surface is in contact and concentric with the eye and wherein the second surface is configured such that incident light on the second surface from an interface of the first surface and the eye does not undergo total internal reflection.

17. A system comprising:
the lens of claim 1; and
the optical attachment of claim 1 comprising a lens arm and a positioning device configured to move the lens and the lens arm relative to the microscope.

18. The system of claim 17 wherein the positioning device is configured to move the lens in contact with an eye of a patient.

19. The system of claim 17, wherein the positioning device comprises an interface for adjustment of the position of the lens relative to the microscope.

20. The system of claim 17, wherein the microscope includes an objective lens that defines an objective optical axis and wherein the positioning device is configured to move the lens along the objective optical axis.

21. A method for using an optical attachment to position a lens relative to a microscope, comprising:
securing the lens to a first end of a lens arm of the optical attachment;
securing a second end of the lens arm to a positioning device of the optical attachment;
moving, with the positioning device, the lens until the lens makes contact with an eye of a patient; and
translating the lens along one or more directions relative to the microscope, based on relative movement between the first end and the second end of the lens arm in the one or more directions such that the lens maintains contact with the eye.

22. The method according to claim 21, wherein the relative movement between the first end and the second end of the lens arm comprises at least one of:
translation between the first end and the second end along the one or more directions to accommodate the translation of the lens along the one or more directions; and
rotation of the first end relative to the second end about one or more rotational axes such that an optical axis of the lens remains oriented within an angular threshold of an optical axis of an objective lens of the microscope during the translating step.

23. The method according to claim 21, wherein the microscope includes an objective lens that defines an objective optical axis;
wherein the securing the second end further comprises securing the positioning device to the microscope;
wherein the moving step comprises moving, with the positioning device, the lens along the objective optical axis until the lens makes contact and is concentric with the eye of the patient;
wherein the method further comprises rotating the lens about one or more rotational axes relative to the microscope based on rotation of the first end of the lens arm relative to the second end of the lens arm such that an optical axis of the lens remains aligned within an angular threshold of the objective optical axis after the translating step.

24. The method according to claim 21, wherein the moving step comprises moving the lens until the lens makes contact with the eye and the lens is within a middle of a range of the translating in the first direction.

25. The method according to claim 23, further comprising pivoting the positioning device, the lens arm and the lens about the objective optical axis.
